(19) United States
(12) Patent Application Publication
(10) Pub. No.: US 2020/0261140 A1
(43) Pub. Date: Aug. 20, 2020

(54) SYSTEM AND METHOD FOR TREATING SOFT TISSUE SARCOMA WITH COLD PLASMA JET

(71) Applicant: JEROME CANADY RESEARCH INSTITUTE FOR ADVANCED BIOLOGICAL AND TECHNOLOGICAL SCIENCES, BENEFIT LLC, Takoma Park, MD (US)

(72) Inventors: Jerome Canady, Lakeland, FL (US); Xiaoqian Cheng, Fairfax, VA (US); Lawan Ly, Annadale, VA (US); Saravana Murthy, Owings Mills, MD (US)

(21) Appl. No.: 16/791,534

(22) Filed: Feb. 14, 2020

Related U.S. Application Data
(60) Provisional application No. 62/806,839, filed on Feb. 17, 2019.

(51) Int. Cl. A61B 18/12; A61B 5/00

(52) U.S. Cl. CPC A61B 18/1206; A61B 2018/00583; A61B 5/748; A61B 5/7435

(57) ABSTRACT

A method for applying cold atmospheric plasma treatment to target tissue comprising the steps of selecting through a graphical user interface a particular soft tissue sarcoma cell line associated with target tissue, retrieving, with said computing device, settings data associated with said selected soft tissue sarcoma cell line from a database of cell line data and associated settings data in a storage, and applying, with said computing device, said retrieved settings data to a cold atmospheric plasma system.

### CROSS-REFERENCE TO RELATED APPLICATIONS

The present application claims the benefit of the filing date of U.S. Provisional Patent Application Ser. No. 62/806,839 filed by the present inventors on Feb. 17, 2019.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field Of The Invention

The present invention relates to systems and methods for treating soft tissue sarcoma with cold atmospheric plasma.

### Brief Description of the Related Art

Soft tissue sarcoma is a malignant tumor that most often develops in adults but can occur in children as well. Treatment with radiation, en bloc surgical resection and chemotherapy have achieved long-term survival rates up to 65% to 80% in non-metastatic disease. Local microscopic tumor cells can still exist despite complete R-0 surgical excision of the tumor.

The unique chemical and physical properties of cold atmospheric plasmas enable their numerous recent applications in biomedicine including sterilization, the preparation of polymer materials for medical procedures, wound healing, tissue or cellular removal and dental drills. A. Fridman, Plasma Chemistry (Cambridge University Press, 2008); G. Fridman, G. Friedman, A. Gutsol, A. B. Shekhter, V. N. Vasilets, and A. Fridman “Applied Plasma Medicine”, Plasma Processes Polym. 5, 503 (2008); E. Stoffels, Y. Sakiyama, and D. B. Graves “Cold Atmospheric Plasma: Charged Species and Their Interactions With Cells and Tissues” IEEE Trans. Plasma Sci. 36, 1441 (2008); X. Lu, Y. Cao, P. Yang, Q. Xiong, Z. Xiong, Y. Xian, and Y. Pan “An RC Plasma Device for Sterilization of Root Canal of Teeth” IEEE Trans. Plasma Sci. 37, 668 (2009).

Plasma-based nitrogen oxide (NO) therapy demonstrated huge potential for stimulation of regenerative processes and wound healing. The work uncovering function of nitrogen oxide as a signal molecule was awarded by the Nobel Prize in medicine and biology in 1999. NO-therapy demonstrated tremendous effect of acceleration of healing of ulcer, burns and serious wounds. Other experimental evidence supports efficiency of cold plasmas produced by dielectric barrier discharge for apoptosis of melanoma cancer cell lines, treatment of cutaneous leishmaniasis, ulcerous eyelid wounds, corneal infections, sterilization of dental cavities, skin regeneration, etc.

Recent progress in atmospheric plasmas led to creation of cold plasmas with ion temperatures close to room temperature. Cold non-thermal atmospheric plasmas can have tremendous applications in biomedical technology. K. H. Becker, K. H. Shoenbach and J. G. Eden “Microplasma and applications” J. Phys. D.: Appl. Phys. 39, R55-R70 (2006). In particular, plasma treatment can potentially offer a minimum-invasive surgery that allows specific cell removal without influencing the whole tissue. Conventional laser surgery is based on thermal interaction and leads to accidental cell death i.e. necrosis and may cause permanent tissue damage. In contrast, non-thermal plasma interaction with tissue may allow specific cell removal without necrosis. In particular, these interactions include cell detachment without affecting cell viability, controllable cell death etc. It can be used also for cosmetic methods of regenerating the reticular architecture of the dermis. The aim of plasma interaction with tissue is not to denaturate the tissue but rather to operate under the threshold of thermal damage and to induce chemically specific response or modification. In particular presence of the plasma can promote chemical reaction that would have desired effect. Chemical reaction can be promoted by tuning the pressure, gas composition and energy. Thus, the important issues are to find conditions that produce effect on tissue without thermal treatment. Overall plasma treatment offers the advantage that is can never be thought of in most advanced laser surgery. E. Stoffels, I. E Kieft, R. E. J Sladek, L. J. M van den Bedem, E. P van der Laan, M. Steinbuch “Plasma needle for in vivo medical treatment: recent developments and perspectives” Plasma Sources Sci. Technol. 15, S169-S180 (2006).

Several different systems and methods for performing Cold Atmospheric Plasma (CAP) treatment have been disclosed. For example, U.S. Published Patent Application No. 2014/0378892 discloses a two-electrode system for CAP treatement. U.S. Pat. No. 9,999,462 discloses a converter unit for using a traditional electrosurgical system with a single electrode CAP accessory to perform CAP treatment.

As a near-room temperature ionized gas, cold atmospheric plasma (CAP) has demonstrated its promising capability in cancer treatment by causing the selective death of cancer cells in vitro. See, Yan D, Sherman J H and Keidar M, “Cold atmospheric plasma, a novel promising anti-cancer treatment modality,” Oncotarget. 8 15977-15995 (2017); Keidar M, “Plasma for cancer treatment,” Plasma Sources Sci. Technol. 24 33001 (2015); Hirst A M, Frame F M, Arya M, Maitland N J and O'Connell D, “Low temperature plasmas as emerging cancer therapeutics: the state of play and thoughts for the future,” Tumor Biol. 37 7021-7031 (2016). The CAP treatment on several subcutaneous xenograft tumors and melanoma in mice has also demonstrated its potential clinical application. See, Keidar M, Walk R, Shashurin A, Srinivasan P, Sandler A, Dasgupta S, Ravi R, Guerrero-Preston R and Trink B, “Cold plasma selectivity and the possibility of a paradigm shift in cancer therapy,” Br. J. Cancer. 105 1295-301 (2011); Vandamme M, Robert E, Dozias S, Sobilo J, Lerondel S, Le Pape A and Pouvesle J-M, “Response of human glioma U87 xenografted on mice to non thermal plasma treatment,” Plasma Med. 1 27-43 (2011); Brulle L, Vandamme M, Ries D, Martel E, Robert E, Lerondel S, Trichet V, Richard S, Pouvesle J M and Le Pape A, “Effects of a Non thermal plasma treatment alone or in combination with gemcitabine in a MIA PaCa2-luc orthotopic pancreatic carcinoma model,” PLoS One. 7 e52653 (2012); and Chernets N, Kurpad D S, Alexeev V, Rodrigues D B and Freeman T A, “Reaction chemistry generated by nanosecond pulsed dielectric barrier discharge treatment is responsible for the tumor eradication in the B16 melanoma mouse model,” Plasma Process. Polym. 12 1400-1409 (2015).

The rise of intracellular reactive oxygen species (ROS), DNA damage, mitochondrial damage, as well as apoptosis have been extensively observed in the CAP-treated cancer cell lines. See, Ahn H J, Kim K Il, Kim G, Moon E, Yang S S and Lee J S, “Atmospheric-pressure plasma jet induces apoptosis involving mitochondria via generation of free radicals,”. PLoS One. 6 e28154 (2011); Ja Kim S, Min Joh H and Chung T H, “Production of intracellular reactive oxygen species and change of cell viability induced by atmospheric pressure plasma in normal and cancer cells,” Appl. Phys. Lett. 103 153705 (2013); and Yan D, Talbot A, Nourmohammadi N, Sherman J H, Cheng X and Keidar M, “Toward understanding the selective anticancer capacity of cold atmospheric plasma—a model based on aquaporins (Review),” Biointerphases. 10 040801 (2015). The increase of intracellular ROS may be due to the complicated intracellular pathways or the diffusion of extracellular ROS through the cellular membrane. See, Yan D, Xiao H, Zhu W, Nourmohammadi N, Zhang L G, Bian K and Keidar M, “The role of aquaporins in the anti-glioblastoma capacity of the cold plasma-stimulated medium,” J. Phys. D. Appl. Phys. 50 055401 (2017). However, the exact underlying mechanism is still far from clear.

Cancer cells have shown specific vulnerabilities to CAP. See, Yan D, Talbot A, Nourmohammadi N, Cheng X, Canady J, Sherman J and Keidar M, “Principles of using cold atmospheric plasma stimulated media for cancer treatment,” Sci. Rep. 5 18339 (2015)

Understanding the vulnerability of cancer cells to CAP will provide key guidelines for its application in cancer treatment. Only two general trends about the cancer cells' vulnerability to CAP treatment have been observed in vitro based on just a few cell lines. First, one study just compared the cytotoxicity of CAP treatment on the cancer cell lines expressing p53 with the same treatment on the cancer cell lines without expressing p53. The cancer cells expressing the p53 gene were shown to be more resistant to CAP treatment than p53 minus cancer cells. Ma Y, Ha C S, Hwang S W, Lee H J, Kim G C, Lee K W and Song K, “Non-thermal atmospheric pressure plasma preferentially induces apoptosis in p53-mutated cancer cells by activating ROS stress-response pathways,” PLoS One. 9 e91947 (2014). p53, a key tumor suppressor gene, not only restricts abnormal cells via the induction of growth arrest or apoptosis, but also protects the genome from the oxidative damage of ROS such as H₂O₂ through regulating the intracellular redox state. Sablina A A, Budanov A V, Ilyinskaya G V, Larissa S, Kravchenko J E and Chumakov P M, “The antioxidant function of the p53 tumor suppressor,” Nat. Med. 11 1306 (2005). p53 is an upstream regulator of the expression of many anti-oxidant enzymes such as glutathione peroxidase (GPX), glutaredoxin 3 (Grx3), and manganese superoxide dismutase (MnSOD). Maillet A and Pervaiz S, “Redox regulation of p53, redox effectors regulated by p53: a subtle balance,” Antioxid. Redox Signal. 16 1285-1294 (2012). In addition, the cancer cells with a lower proliferation rate are more resistant to CAP than cancer cells with a higher proliferation rate. Naciri M, Dowling D and Al-Rubeai M, “Differential sensitivity of mammalian cell lines to non-thermal atmospheric plasma,” Plasma Process. Polym. 11 391-400 (2014). This trend may be due to the general observation that the loss of p53 is a key step during tumorigenesis. Tumors at a high tumorigenic stage are more likely to have lost p53. See, Fearon E F and Vogelstein B, “A genetic model for colorectal tumorigenesis,” Cell. 61 759-767 (1990).

Despite the complicated interaction between CAP and cancer cells, the initial several hours after treatment has been found to be an important stage for the cytotoxicity of CAP. The anti-cancer ROS molecules in the extracellular medium are completely consumed by cells during this time period. After the initial several hours, replacing the medium surrounding the cancer cells does not change the cytotoxicity of CAP. See, Yan D, Cui H, Zhu W, Nourmohammadi N, Milberg J, Zhang L G, Sherman J H and Keidar M, “The specific vulnerabilities of cancer cells to the cold atmospheric plasma-stimulated solutions,” Sci. Rep. 7 4479 (2017).

### SUMMARY OF THE INVENTION

Cold atmospheric plasma (CAP) reduces sarcoma cell viability in a time- and power-dependent manner. With optimal dosage for each cancer type, soft tissue sarcomas may be treated effectively.

Soft tissue sarcomas can be tested to provide a rough prediction of the most effective time and power settings for CAP treatment. The results of this testing may be used to generate a database of soft tissue sarcomas with associated predicted optimum setting or dosage data and optionally effectiveness data. This database can be stored in memory or other storage in a CAP capable electrosurgical system or can be in an external storage that can be accessed by a CAP capable electrosurgical system. The CAP capable electrosurgical system may have a user interface that then allows a user to enter an identifier for a particular cancer cell line into the user interface and thereby have the CAP enabled electrosurgical system automatically select the predicted optimum settings or dosage for that particular cancer cell line. The user can then perform a CAP treatment of target cancer cells at those predicted optimum settings.

In a preferred embodiment, the present invention is a method for applying cold atmospheric plasma treatment to target tissue. The method comprises selecting through a graphical user interface a particular soft tissue sarcoma cell line associated with target tissue, retrieving, with the computing device, settings data associated with the selected soft tissue sarcoma cell line from a database of cell line data and associated settings data in a storage, and applying, with the computing device, the retrieved settings data to a cold atmospheric plasma system.

In another preferred embodiment, the method comprises generating a database of a plurality of soft tissue sarcoma cell lines and optimum cold atmospheric plasma settings associated with each of the plurality of soft tissue sarcoma cell lines, storing the database in a storage medium, selecting through a graphical user interface on a computing device a particular soft tissue sarcoma cell line associated with the target tissue, retrieving, with the computing device, settings data from a database of cell line data and associated settings data in a storage, and applying, with the computing device, the retrieved settings data to a cold atmospheric plasma system. Further, the cold atmospheric plasma settings in the generated database may be based upon a predicted CAP effectiveness derived from testing the plurality of soft tissue sarcoma cells lines with CAP treatment at a plurality of settings.

In still another embodiment, the present invention is a system for applying cold atmospheric plasma treatment to target tissue. The system has a source of an inert gas, a source of electrosurgical energy, a gas control module connected to the source of an inert gas, a processor configured to control the gas control module and the source of electrosurgical energy, an electronic storage medium connected to the processor, a database stored in the storage medium, the database comprising identifying information of each of a plurality of soft tissue sarcoma cell lines and optimum cold atmospheric plasma settings associated with each of the plurality of soft tissue sarcoma cell lines a graphical user interface connected to the processor, the graphical user interface configured to provide for a user input of a selected soft tissue sarcoma cell line, and an applicator connected to the gas control module and the source of electrosurgical energy for applying cold atmospheric plasma to target tissue. The processor is configured to retrieve from the database cold atmospheric plasma settings associated with a soft tissue sarcoma cell line selected through the graphical user interface in response to the user input and to control the gas control module and the source of electrosurgical energy.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1A is a perspective view of a preferred embodiment of a gas-enhanced electrosurgical generator.

FIG. 1B is a front view of a preferred embodiment of a gas-enhanced electrosurgical generator.

FIG. 1C is a rear view of a preferred embodiment of a gas-enhanced electrosurgical generator.

FIG. 1D is a left side view of a preferred embodiment of a gas-enhanced electrosurgical generator.

FIG. 1E is a right view of a preferred embodiment of a gas-enhanced electrosurgical generator.

FIG. 1F is a top view of a preferred embodiment of a gas-enhanced electrosurgical generator.

FIG. 1G is a bottom view of a preferred embodiment of a gas-enhanced electrosurgical generator.

FIG. 2A is a block diagram of a preferred embodiment of pressure control system of a gas-enhanced electrosurgical generator in accordance with the present invention configured to perform an argon-enhanced electrosurgical procedure.

FIG. 2B is a block diagram of a preferred embodiment of pressure control system of a gas-enhanced electrosurgical generator in accordance with the present invention configured to perform a cold atmospheric plasma procedure.

FIG. 2C is a diagram of a trocar for the embodiment of FIG. 2A in accordance with the present invention.

FIG. 2D is a block diagram of an alternate preferred embodiment of pressure control system of a gas-enhanced electrosurgical generator in accordance with the present invention configured to perform an argon-enhanced electrosurgical procedure.

FIG. 3A is a schematic flow diagram illustrating the gas flow through the module and the method by which the module controls the gas flow in accordance with a preferred embodiment of the present invention.

FIG. 3B is a schematic flow diagram illustrating the gas flow through an alternate embodiment of the module and the method by which the module controls the gas flow in accordance with a preferred embodiment of the present invention.

FIG. 4 is a diagram of a graphical user interface in accordance with a preferred embodiment of the present invention.

FIG. 5 is a graph illustrating viability of synovial sarcoma 48 hours post CAP treatment.

FIG. 6 is a graph illustrating viability of connective tissue fibro sarcoma 48 hours post CAP treatment.

FIG. 7 is a graph illustrating viability of rhabdomyosarcoma 48 hours post CAP treatment.

FIG. 8 is a flow chart of a method for performing CAP treatment in accordance with a preferred embodiment of the present invention.

### DETAILED DESCRIPTION OF THE PREFERRED EMBODIMENTS

As shown in the experiments discussed below, soft tissue sarcomas can be tested at varying settings or dosages of the CAP treatment to provide an estimate of which CAP treatment settings or dosages will provide the greatest effect on particular cell lines. In a preferred embodiment of the present invention, the results of such testing are used to generate a database of cancer cell lines with associated predicted optimum settings or dosage data and optionally effectiveness data. This database can be stored in memory or other storage in a CAP capable electrosurgical system or can be in an external storage, for example, accessible through a server or cloud computing system, that can be accessed by a CAP capable electrosurgical system. The CAP capable electrosurgical system may have a graphical user interface that allows a user to enter an identifier for a particular cancer cell line into the user interface and thereby have the CAP enabled electrosurgical system automatically select the predicted optimum settings or dosage for that particular cancer cell line. The user can then perform a CAP treatment of target cancer cells at those predicted optimum settings.

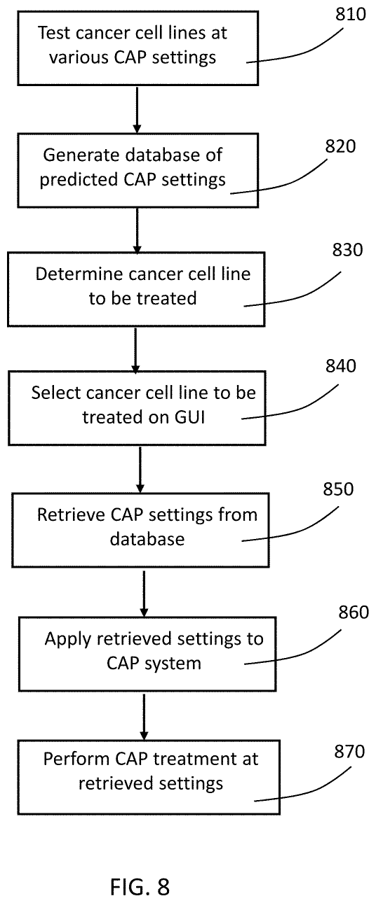

Thus, as shown in FIG. 8, a method can be performed in which a plurality of cancer cell lines can be tested (step 810) to determine optimal treatment settings for that cell line. A database of cancer cell lines and associated CAP treatment data is generated and stored, for example in storage 212 or in memory in a processor, microcontroller or CPU 210 or other memory or electronic storage device (step 820). The data for generating the database can be entered through an input means such as the graphical user interface on the generator or through an input means connected, for example, to a server, or can be uploaded into the generator or storage through an I/O port on the generator. Additional cancer cell line data and associate settings or dosage data can be added to the database in the storage as new cell lines are tested and new data is developed. Such additional data can be entered via the same means described above. If the generator is connected, for example, to a wireless network or to a portable computer or tablet, the data similarly can be downloaded through such a wireless network (or through a Bluetooth connection) to the generator 200 and/or the storage 212. A user performing a CAP treatment determines the particular cancer cell line to be treated (step 830) and then enters an identifier associated with a particular cancer cell line into a graphical user interface on a CAP capable electrosurgical system (step 840). The CAP capable electrosurgical system then accesses the stored database to retrieve CAP setting or dosages associated with the ID entered into the graphical user interface (step 850). The phrase “enter an identifier” used herein can mean any data entry or selection by the user that provides the graphical user interface with sufficient information to retrieve data from the database for a particular cell line. This could be selection from a list or menu, entry of an identifier through a physical or virtual keyboard associated with the system, scanning of a bar code, or any other means. Further, the graphical user interface and associated display do not need to physically be in the CAP capable generator but instead may be on external devices such as a tablet computing device that is in communication with the CAP enabled electrosurgical generator. The retrieved CAP settings are then applied to the CAP system (step 860). The user then can treat the target tissue with CAP at the preferred settings (step 870).

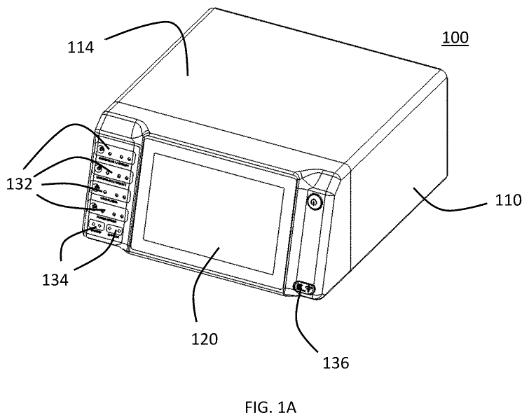

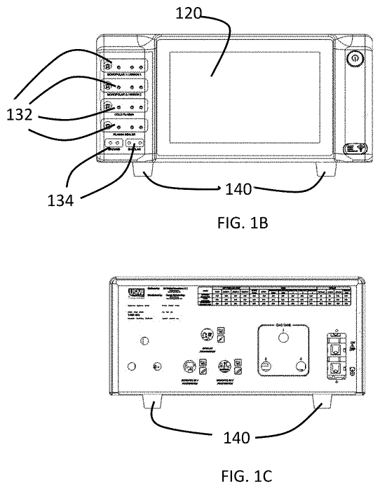

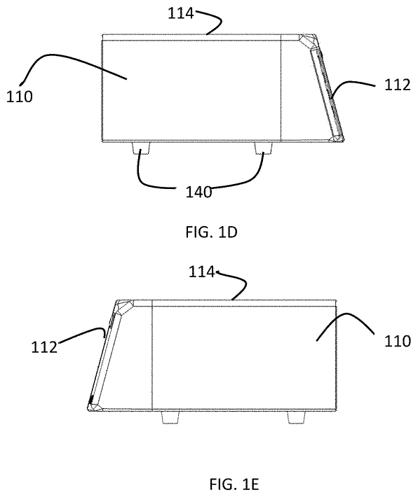

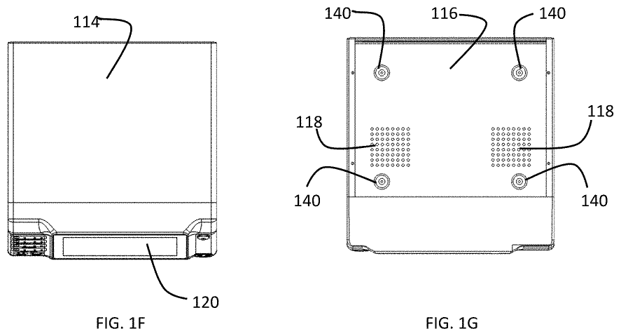

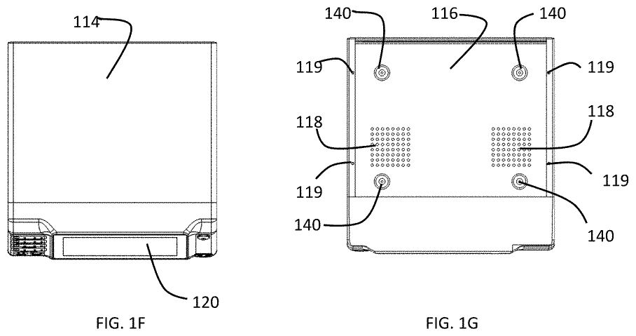

A preferred embodiment of a CAP enabled generator is described with reference to the drawings. A gas-enhanced electrosurgical generator 100 in accordance with a preferred embodiment of the present invention is shown in FIGS. 1A-1G. The gas-enhanced generator has a housing 110 made of a sturdy material such as plastic or metal similar to materials used for housings of conventional electrosurgical generators. The housing 110 has a removable cover 114. The housing 110 and cover 114 have means, such as screws 119, tongue and groove, or other structure for removably securing the cover to the housing. The cover 114 may comprise just the top of the housing or multiple sides, such as the top, right side and left side, of the housing 110. The housing 110 may have a plurality of feet or legs 140 attached to the bottom of the housing. The bottom 116 of the housing 110 may have a plurality of vents 118 for venting from the interior of the gas-enhanced generator.

On the face 112 of the housing 114 there is a touch-screen display 120 and a plurality of connectors 132, 134 for connecting various accessories to the generator, such as an argon plasma probe, a hybrid plasma probe, a cold atmospheric plasma probe, or any other electrosurgical attachment. There is a gas connector 136 for connecting, for example, a CO₂ supply for insufflating an abdomen. The face 112 of the housing 110 is at an angle other than 90 degrees with respect to the top and bottom of the housing 110 to provide for easier viewing and use of the touch screen display 120 by a user.

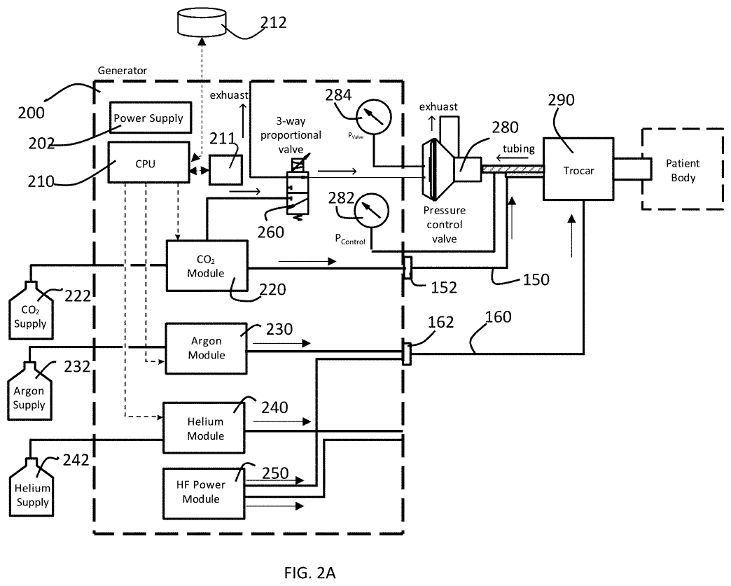

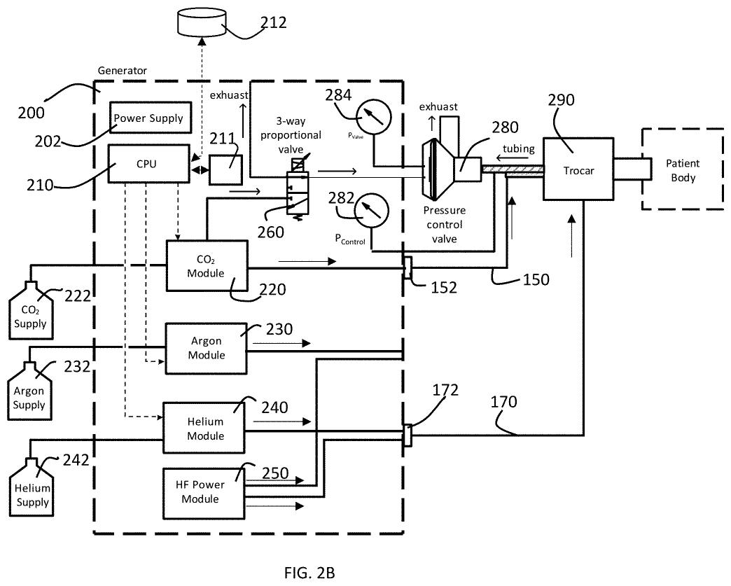

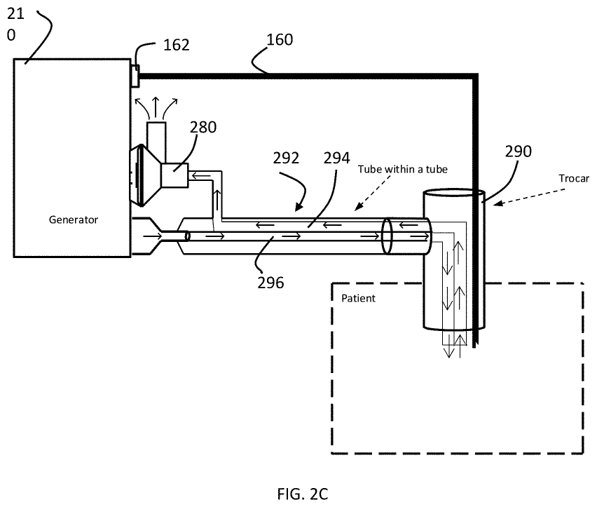

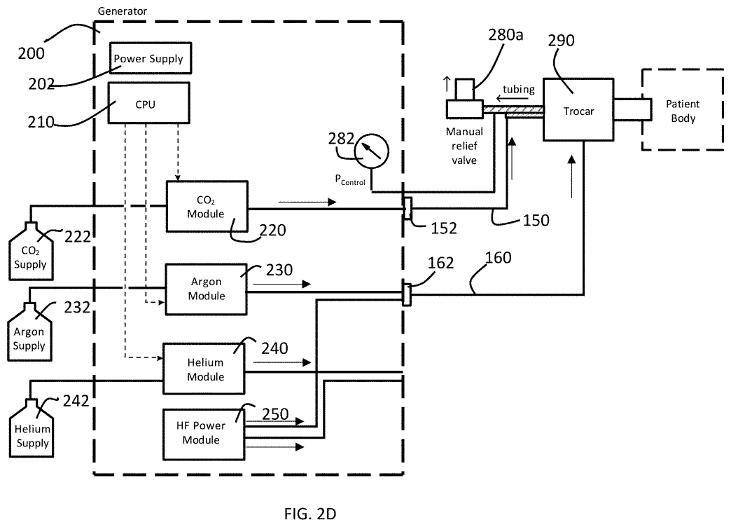

One or more of the gas control modules may be mounting within a gas-enhanced electrosurgical generator 100. A gas pressure control system 200 for controlling a plurality of gas control modules 220, 230, 240 within a gas-enhanced electrosurgical generator is described with reference to FIGS. 2A-2D. A plurality of gas supplies 222, 232, 242 are connected to the gas pressure control system 200, and more specifically, to the respective gas control modules 220, 230, 240 within the gas pressure control system 200. The gas pressure control system 200 has a power supply 202 for supplying power to the various components of the system. A CPU 210 controls the gas pressure control modules 220, 230, 240 in accordance with settings or instructions entered into the system through a graphical user interface on the display 120. The system is shown with gas control modules for CO₂, argon and helium, but the system is not limited to those particular gases. In the embodiment shown in FIGS. 2A-2D, the CO₂ is shown as the gas used to insufflate an abdomen (or other area of a patient). The gas pressure control system 200 has a 3-way proportional valve connected to the gas control module 220. While FIG. 2A shows the 3-way proportional valve connected only to the CO₂ control module 220, the 3-way proportional valves could be connected to a different gas control module 230 or 240. The gas pressure control system 200 further has an HF power module 250 for supplying high frequency electrical energy for various types of electrosurgical procedures. The HF power module contains conventional electronics such as are known for provide HF power in electrosurgical generators. Exemplary systems include, but are not limited to, those disclosed in U.S. Pat. Nos. 4,040,426 and 4,781,175. The system further could have a converter unit for converting the HF power to a lower frequency, such as may be used for cold atmospheric plasma and is described in U.S. Patent Application Publication No. 2015/0342663. As used herein, the term “high frequency” means a frequency above 300 Hz and “low frequency” means a frequency less than 300 Hz.

The outlet port of gas control module 220 is connected to a connector 136 on the generator housing. While connector 136 and the other connectors are shown on the front face of the housing 110, they could be elsewhere on the housing. The outlet ports of gas control modules 230, 240 each are connected to tubing or other channel to a connector 132. A connector 152 connects to connector 136 and is as tubing that runs to and connects to tubing 292. The tubing 292 is connected to a pressure control valve or stopcock 280 and extends into the trocar. The pressure control valve 280 is used to control pressure within the patient. The gas pressure control system further has a pressure sensor 282 connected to the tubing 292 to sense pressure in the tubing 292 and a pressure sensor 284 for sensing pressure in the pressure control valve 280. As shown in FIG. 2C, the tubing 292 is a tube within a tube such that gas supplied from the generator travels to the trocar and patient through tube 296 and gas is released out of the patient through tube 294.

As shown in FIG. 2A the connector 132 to which control module 230 is connected has a gas-enhanced electrosurgical instrument 160 having a connector 162 connected to in. In FIG. 2A, gas control module 230 controls flow of argon gas, so the instrument 160 is an argon gas-enhanced electrosurgical tool such as an argon plasma probe such as is disclosed in U.S. Pat. No. 5,720,745, a hybrid plasma cut accessory such as is disclosed in U.S. Patent Application Publication No. 2017/0312003 or U.S. Patent Application Publication No. 2013/0296846, or a monopolar sealer such as is disclosed in U.S. Patent Application Publication No. 2016/0235462. Other types of argon surgical devices similarly can be used. As shown in FIG. 2B the connector 132 to which control module 240 is connected has a gas-enhanced electrosurgical instrument 170 having a connector 172 connected to in. In FIG. 2B, gas control module 240 controls flow of helium gas, so the instrument 170 is, for example, a cold atmospheric plasma attachment such as is disclosed in U.S. Patent Application Publication No. 2016/0095644.

The system provides for control of intraabdominal pressure in a patient. The pressure control valve 280 has a chamber within it. The pressure in that chamber is measured by pressure sensor 284. CO₂ is supplied to the chamber within pressure control valve 280 from gas control module 220 via 3-way proportional valve 260. Pressure in that chamber within the pressure control valve 280 also may be released via 3-way proportional valve 260. In this manner, the system can use the pressure sensor 284 and the 3-way proportional valve to achieve a desired pressure (set through a user interface) in the chamber within the pressure control valve 280. The pressure sensor 282 senses the pressure in the tubing 294 (and hence the intraabdominal pressure). The pressure control valve 280 then releases pressure through its exhaust to synchronize the intraabdominal pressure read by sensor 282 with the pressure in the chamber within the pressure control valve as read by pressure sensor 284. The readings from sensors 282, 284 can be provided to CPU 210, which in turn can control flow of CO₂ and one of argon and helium, depending on the procedure being performed, to achieve a stable desired intraabdominal pressure.

An alternative embodiment of the gas pressure control system is shown in FIG. 2D. This this system the automatic stopcock or pressure control valve 280 has been replaced by a manual relief valve 280a that is controlled or operated by the surgeon using the system.

A gas control module 300 in accordance with the present invention is designed for gas-enhanced electrosurgical systems. Conventionally, gas-enhanced electrosurgical systems have an electrosurgical generator and a gas control unit that have separate housings. The conventional gas control unit typically controls only a single gas such as argon, CO₂ or helium. The present invention is a gas control module 300 that may be used in a gas control unit or in a combined unit functioning both as an electrosurgical generator and as a gas control unit. Further, a plurality of gas control modules in accordance with the present invention may be combined in a single gas control unit or combination generator/gas control unit to provide control of multiple gases and provide control for multiple types of gas-enhanced surgery such as argon gas coagulation, hybrid plasma electrosurgical systems and cold atmospheric plasma systems.

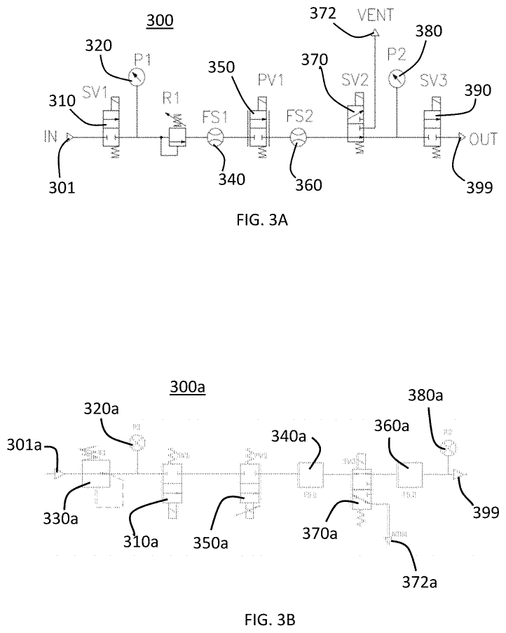

FIG. 3A is a schematic flow diagram illustrating the gas flow through the gas control module 300 and the method by which the module 300 controls the gas flow in accordance with a preferred embodiment of the present invention. As shown in FIG. 3A, the gas enters the gas control module at an inlet port (IN) 301 and proceeds to first solenoid valve (SV1) 310, which is an on/off valve. In an exemplary embodiment, the gas enters the gas module at a pressure of 75 psi. The gas then proceeds to a first pressure sensor (P1) 320, to a first pressure regulator (R1) 330. In an exemplary embodiment, the first pressure regulator (R1) 330 reduces the pressor of the gas from 75 psi to 18 psi. After the pressure regulator (R1) 330, the gas proceeds to flow sensor (FS1) 340, which sense the flow rate of the gas. Next, the gas proceeds to proportional valve (PV1) 350, which permits adjustment of a percentage of the opening in the valve. The gas then proceeds to a second flow sensor (FS2) 360, which senses the flow rate of the gas. This second flow sensor (FS2) 360 provides redundancy and thus provides greater safety and accuracy in the system. Next the gas proceeds to a second solenoid valve (SV2) 370, which is a three-way valve that provides for a vent function that can allow gas to exit the module through a vent 372. The gas then proceeds to a second pressure sensor (P2) 380, which provides a redundant pressure sensing function that against produces greater safety and accuracy of the system. Finally, the gas proceeds to a third solenoid valve (SV3) 390, which is a two-way on/off valve that is normally closed and is the final output valve in the module. The gas exits the module at and output port (OUT) 399, which is connected to tubing or other channel that provides a passageway for the gas to flow to an accessory connected to the electrosurgical unit.

FIG. 3B is a schematic flow diagram of an alternate embodiment of a gas control module illustrating the gas flow through the gas control module 300a and the method by which the module 300a controls the gas flow in accordance with a preferred embodiment of the present invention. As shown in FIG. 3B, the gas enters the gas control module at an inlet port 301a and proceeds to a first pressure regulator (R1) 330a. In an exemplary embodiment, the first pressure regulator (R1) 330a reduces the pressor of the gas from about 50-100 psi to 15-25 psi. After the pressure regulator (R1) 330a, the gas proceeds to a first pressure sensor (P1) 320a and then to a first solenoid valve (SV1) 310a, which is an on/off valve. Next, the gas proceeds to proportional valve (PV1) 350a, which permits adjustment of a percentage of the opening in the valve. Next, the gas proceeds to flow sensor (FS1) 340a, which sense the flow rate of the gas. ext the gas proceeds to a second solenoid valve (SV2) 370a, which is a three-way valve that provides for a vent function that can allow gas to exit the module through a vent 372a. The gas then proceeds to a second flow sensor (FS2) 360a, which senses the flow rate of the gas. This second flow sensor (FS2) 360a provides redundancy and thus provides greater safety and accuracy in the system. The gas then proceeds to a second pressure sensor (P2) 380a, which provides a redundant pressure sensing function that against produces greater safety and accuracy of the system. The gas exits the module at and output port 399a, which is connected to tubing or other channel that provides a passageway for the gas to flow to an accessory connected to the electrosurgical unit.

The various valves and sensors in either embodiment of the module are electrically connected to a main PCB Board through a connector 490. The PCB connector 490 is connected to a PCB Board that has a microcontroller (such as CPU 210 in the embodiment shown in FIG. 2A). As previously noted, a plurality of gas modules can be in a single gas control unit or single electrosurgical generator to provide control of multiple differing gases. The plurality of gas control modules further may be connected to the same PCB Board, thus providing common control of the modules.

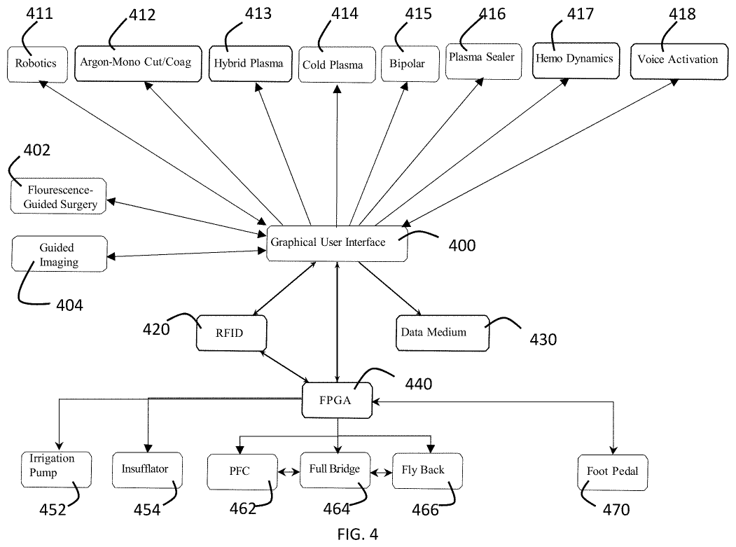

As shown in FIG. 4, the generator further may have graphical user interface 400 for controlling the components of the system using the touch screen display 120. The graphical user interface 400 for example, may control robotics 411, argon-monopolar cut/coag 412, hybrid plasma cut 413, cold atmospheric plasma 414, bipolar 415, plasma sealer 416, hemo dynamics 417 or voice activation 418. The graphical user interface further may be used with fluorescence-guided surgery 402. For example, J. Elliott, et al., “Review of fluorescence guided surgery visualization and overlay techniques,” BIOMEDICAL OPTICS EXPRESS 3765 (2015), outlines five practical suggestions for display orientation, color map, transparency/alpha function, dynamic range compression and color perception check. Another example of a discussion of fluorescence-guided surgery is K. Tipirneni, et al., “Oncologic Procedures Amenable to Fluorescence-guided Surgery,” Annals of Surgery, Vo. 266, No. 1, July 2017). The graphical user interface (GUI) further may be used with guided imaging such as CT, MRI or ultrasound. The graphical user interface may communicate with RFID 420 (such as may be found in various electrosurgical attachments) and may collect and store usage data in a storage medium 430. The graphical user interface 400 communicates with FPGA 440, which may control irrigation pump 452, insufflator 454, PFC 462, full bridge 464 for adjusting the power output, fly back 466 for regulating the power (DC to AC) and a foot pedal 470. The GUI 400 further communicates with a database of cancer cell line data with associated predicted CAP settings or dosages via the CPU 210. The databases storage may be internal memory or other internal storage 211 or external storage 212 as shown in FIGS. 2A and 2B. The data storage 430 in FIG. 4 may be in one or both memories or storages 211 or 212.

### EXPERIMENTS

CAP was generated using a US Medical Innovations LLC (USMI) SS-601 MCa high-frequency electrosurgical generator (USMI, Takoma Park, Md., USA) integrated with Canady Cold Plasma Conversion Unit and connected to a Canady Helios Cold Plasma Scalpel. Three types of human sarcoma cells, synovial sarcoma (SW982), connective tissue fibro sarcoma (HT-1080), and rhabdomyosarcoma (RD) were used in this experiment to test the effect of the CAP generated by the Canady Cold Plasma Conversion System. Cells were treated with various CAP settings including different helium flow rates (1 and 3 LPM) and power settings (20-120 p) in order to establish an optimal treatment condition for each cell line. Viability was performed on the cells using MTT assay 48 hours after CAP treatment. Student t test was performed on the data (*p<0.05).

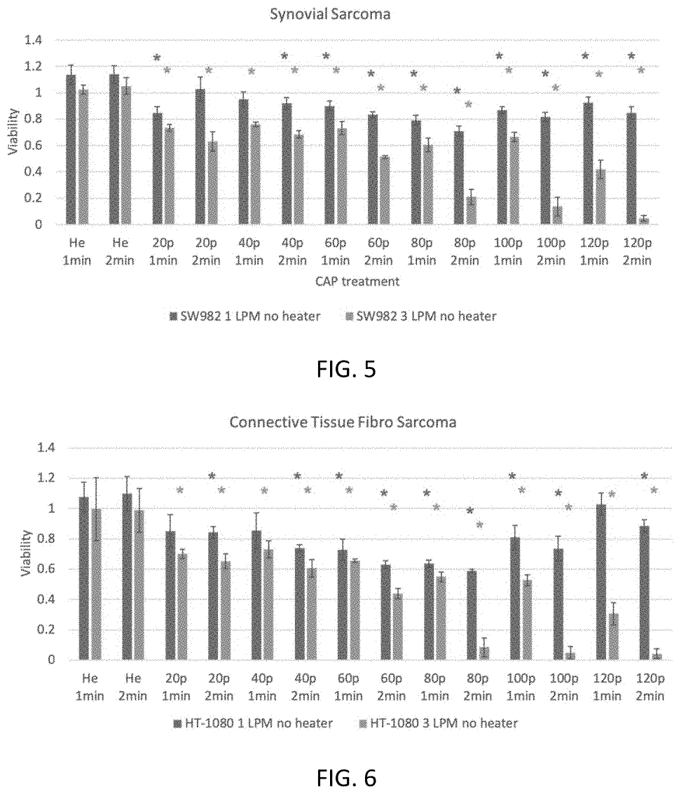

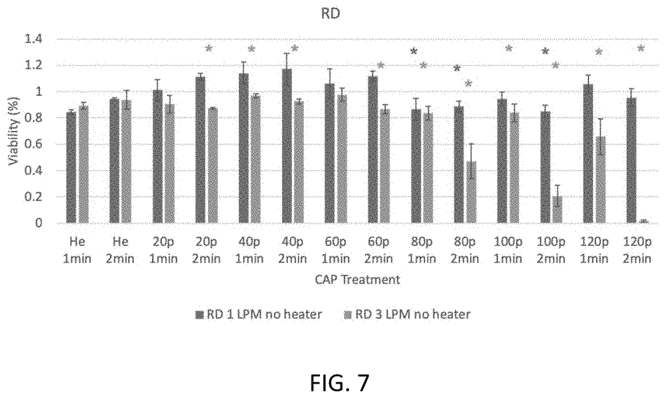

The reduction of the viability of all three sarcomas were dose-dependent and significantly reduced at various time and power combinations tested (FIGS. 5-7). Helium flow alone did not significantly impact cell viability. The decrease in viability of the sarcoma cells when using 1 LPM required a higher dose. About 20 to 40% of viability reduction was seen on the three cell lines. With 3 LPM, viability was reduced to 20% using 80 p 2 min for SW982 and HT-1080, and 100 p 2 min for RD.

The experimental data demonstrates that CAP reduced sarcoma cell viability in a time- and power-dependent manner. With optimal dosage for each cancer type, the present invention provides a promising treatment for future therapeutic interventions for soft tissue sarcomas.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for applying cold atmospheric plasma treatment to target tissue comprising:
selecting through a graphical user interface on a display connected to a computing device a particular soft tissue sarcoma cell line associated with target tissue;
retrieving, with said computing device, settings data associated with said selected soft tissue sarcoma cell line from a database of cell line data and associated settings data in a storage; and
applying, with said computing device, said retrieved settings data to a cold atmospheric plasma system.

2. A method for applying cold atmospheric plasma treatment to target tissue according to claim 1, wherein said display is a display of a tablet computer.

3. A method for applying cold atmospheric plasma treatment to target tissue according to claim 1, wherein said settings data comprises at least one of a power setting a flow rate setting and a time setting.

4. A method for applying cold atmospheric plasma treatment to target tissue according to claim 1, further comprising treating cancerous tissue with said cold atmospheric plasma system with said applied retrieved settings data.

5. A method for applying cold atmospheric plasma treatment to target tissue comprising:
generating a database of a plurality of soft tissue sarcoma cell lines and optimum cold atmospheric plasma settings associated with each of said plurality of soft tissue sarcoma cell lines;
storing said database in an electronic storage medium;
selecting through a graphical user interface on a computing device a particular soft tissue sarcoma cell line associated with said target tissue;
retrieving from said electronic storage medium, with said computing device, optimum cold atmospheric plasma settings data associated with said particular soft tissue sarcoma cell line from said database; and
applying, with said computing device, said retrieved settings data to a cold atmospheric plasma system.

6. A method for applying cold atmospheric plasma treatment to target tissue according to claim 5, wherein said optimum cold atmospheric plasma settings in said generated database are based upon a predicted CAP effectiveness derived from testing said plurality of soft tissue sarcoma cells lines with CAP treatment at a plurality of settings.

7. A system for applying cold atmospheric plasma treatment to target tissue comprising:
a source of electrosurgical energy;
a gas control module connected to a source of an inert gas;
a processor configured to control said gas control module and said source of electrosurgical energy;
an electronic storage medium connected to said processor;
a database stored in said storage medium, said database comprising identifying information of each of a plurality of soft tissue sarcoma cell lines and optimum cold atmospheric plasma settings associated with each of said plurality of soft tissue sarcoma cell lines;
a graphical user interface connected to said processor, said graphical user interface configured to provide for a user input of a selected soft tissue sarcoma cell line; and
an applicator connected to said gas control module and said source of electrosurgical energy for applying cold atmospheric plasma to target tissue;
wherein, said processor is configured to retrieve from said database cold atmospheric plasma settings associated with a soft tissue sarcoma cell line selected through said graphical user interface in response to said user input and to control said gas control module and said source of electrosurgical energy.

8. A system for applying cold atmospheric plasma treatment to target tissue according to claim 7, further comprising:
3-way proportional valve connected to the gas control module.

9. A system for applying cold atmospheric plasma treatment to target tissue according to claim 7, wherein said gas control module comprises:
an inlet port;
a first solenoid valve connected to said inlet port, said first solenoid valve being configured to turn a flow of gas into the gas control module on and off;
a first pressure sensor configured to sense a first pressure of gas entering the gas control module through the first solenoid valve;
a first pressure regulator configured to change said first pressure of gas entering said first pressure regulator to a second pressure;
a first flow sensor configured to sense a flow rate of gas exiting said first pressure regulator;
a first proportional valve having an inlet and an outlet, said first proportional valve being configured to adjust said outlet as a percentage of said inlet;
a second flow sensor configured to sense a flow of gas exiting said first proportional valve;
a second solenoid valve, said second solenoid valve being a 3-way valve;
a vent connected to said second solenoid valve;
a second pressure sensor for sensing a pressure of gas passing through said second solenoid valve; and
a third solenoid valve, said third solenoid valve being configured to turn a flow of gas out of the gas control module on and off; and
an exit port.

10. A system for applying cold atmospheric plasma treatment to target tissue according to claim 7, wherein said a source of electrosurgical energy comprises:
a high frequency power module for supplying high frequency electrical energy for various types of electrosurgical procedures; and
a low frequency converter for converting electrical energy from said high frequency power module to low frequency energy.
